(19) United States
(12) Patent Application Publication
(10) Pub. No.: US 2014/0228590 A1
(43) Pub. Date: Aug. 14, 2014

(54) SIMPLIFIED RADIOSYNTHESIS OF [18F]FLUOROMETHYL BROMIDE

(71) Applicant: Keith Graham, Berlin (DE)

(72) Inventor: Keith Graham, Berlin (DE)

(73) Assignee: PIRAMAL IMAGING SA, MATRAN (CH)

(21) Appl. No.: 14/239,796

(22) PCT Filed: Aug. 27, 2012
(86) PCT No.: PCT/EP2012/066621

§ 371 Date: Apr. 28, 2014

(30) Foreign Application Priority Data
Aug. 25, 2011 (EP) 11178907.9

(51) Int. Cl. C07C 17/383; B65D 77/08; C07C 303/44

(52) U.S. Cl. CPC C07C 17/383; C07C 303/44; B65D 77/08; C07B 2200/05
USPC 558/53; 570/178; 206/568

(57) ABSTRACT

This invention relates to an improved radiosynthesis of [¹⁸F]fluoromethyl bromide, whereby the distillation step has been improved to remove higher amounts of the dibromomethane radiolabeling precursor.

### FIELD OF INVENTION

This invention relates to an improved radiosynthesis of [¹⁸F]fluoromethyl bromide, whereby the distillation step has been improved to remove higher amounts of the dibromomethane radiolabeling precursor.

### BACKGROUND

The invention relates to the subject matter referred to in the claims, i.e. an improved radiosynthesis of [¹⁸F]fluoromethyl bromide ([¹⁸F]FCH₂Br).

Molecular imaging has the potential to detect disease progression or therapeutic effectiveness earlier than most conventional methods in the fields of oncology, neurology and cardiology. Of the several promising molecular imaging technologies having been developed as optical imaging and MRI, PET is of particular interest for drug development because of its high sensitivity and ability to provide quantitative and kinetic data.

Positron emitting isotopes include carbon, nitrogen, and oxygen. These isotopes can replace their non-radioactive counterparts in target compounds to produce tracers that function biologically and are chemically identical to the original molecules for PET imaging. On the other hand, ¹⁸F is the most convenient labeling isotope due to its relatively long half life (109.6 min), which permits the preparation of diagnostic tracers and subsequent study of biochemical processes. In addition, its high β+ yield and low β+ energy (635 keV) are also advantageous.

Due to its short 20 minutes half-life ¹¹C containing radiotracers require an on-site cyclotron, whereas ¹⁸F PET tracers, considering a half-life of 109 minutes, allow for off-site production and regional distribution.

The radiosyntheses of numerous [¹⁸F]-labeled PET tracers are typically carried out via a two-step indirect method, whereby a precursor is radiolabeled with fluorine-18 to give an [¹⁸F]-radiolabeled intermediate (also known as a prosthetic group), which is purified and reacted further with a biological active targeting molecule to give the desired [¹⁸F]-labeled PET tracer; the general approach described above is outlined in Scheme 1.

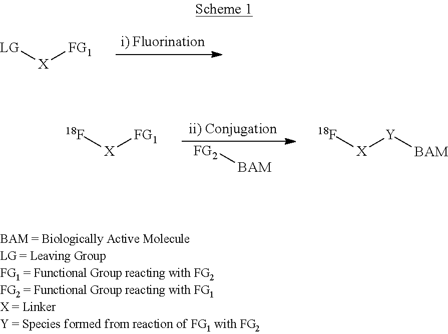

The [¹⁸F]-labeled prosthetic group ([¹⁸F]F-X-FG₁) can include various different functional groups and are listed in the ‘Reference Book for PET Radiopharmaceuticals’ by Ren Iwata (4^{th} Version 2004). Below are listed some selected examples with literature references:
i) [¹⁸F]-labeled activated esters: to react with nucleophiles e.g. amines. Selected examples of [¹⁸F]-labeled activated esters are: succinimidyl 4-[¹⁸F]fluorobenzoate (SFB, Guhlke et al., Appl. Radiat. Isot., 1992, 43, 1335-1339) and succinimidyl 4-([¹⁸F]fluoromethyl)benzoate (Lang et al., Appl. Radiat. Isot., 1994, 45, 1155-1163.
ii) [¹⁸F]-labeled amines: to react with electrophiles, e.g. activated esters. Selected examples of [¹⁸F]-labeled amines are: 4-[¹⁸F]fluoroaniline (Shiue et al., J. Labelled. Radiopharm. Cmpds., 1984, 21, 533-547) and 4-([¹⁸F]fluorobenzyl amine (Koslowsky et al., Org. Biomol. Chem., 2010, 8, 4730-4735).
iii) [¹⁸F]-labeled maleimides: to react with thiols. Selected example of a [¹⁸F]-labeled maleimides is N-[4-[(4-[¹⁸F]fluorobenzylidene)aminooxy]butyl]maleimide (FBABM, Li et al., Bioconjugate Chem., 2008, 19, 1684-1688).
iv) [¹⁸F]-labeled azides: to react with alkynes or Staudinger reagents. Examples of these reactions are reviewed (for reaction with alkynes see the review; Ross., Current Radiopharmaceuticals, 2010, 3, 202-223; for the Staudinger reaction see Pretze et al., Tetrahedron Lett., 2010, 51, 6410-6414).
v) [¹⁸F]-labeled alkynes: to react with azides. Examples of these reactions are reviewed (see Ross., Current Radiopharmaceuticals, 2010, 3, 202-223).

These listed methods only highlight a few of the different methods that have been used to radiolabel different biologically active molecules with fluorine-18. However, the most widely used method is the simple alkylation reaction of nucleophiles, e.g. amines, thiols, phenols, etc. There have been numerous different alkylating agents and a few of these are illustrated in Scheme 2.

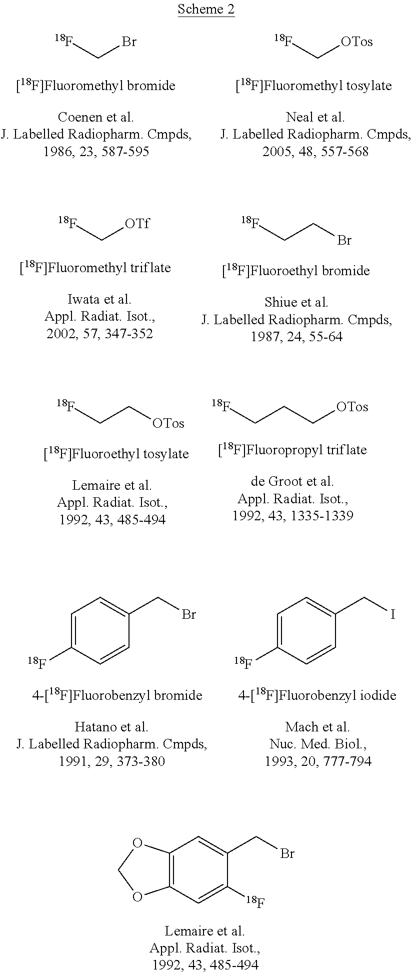

Of these different alkylating agents the fluoromethyl alkylation reaction is of particular interest; especially since the emergence of D-[¹⁸F]fluoromethyl tyrosine (DFMT), an interesting radiolabeled amino acid, which shows promise as a PET tracer for tumor imaging (Tsukada et al., J. Nucl. Med., 2006, 47, 679, Tsukada et al., Eur. J. Nucl. Med. Mol. Imaging, 2006, 33, 1017 and Urakami et al., Nucl. Med. Biol., 2009, 36, 295). This [¹⁸F]fluoromethyl alkylation reaction has also been used for numerous other [¹⁸F]-labeled tracers (Scheme 3): [¹⁸F]fluorocholine (Iwata et al., Appl. Radiat. Isot., 2002, 57, 347-352), [¹⁸F]DFMT (Tsukada et al., J. Nucl. Med., 2006, 47, 679), (S,S)-[¹⁸F]FMeNER (Synapse, Schou et al., 2004, 53, 57-67), [¹⁸F]FMDAA (Zhang et al., J. Med. Chem., 2004, 47, 2228-2235), [¹⁸F]SPA-RQ (Chin et al., J. Labelled Radiopharm. Cmpds., 2006, 49, 17-31) and [¹⁸F]fluticasone propionate ([¹⁸F]FP, Neal et al., J. Labelled Radiopharm. Cmpds., 2005, 48, 557-568).

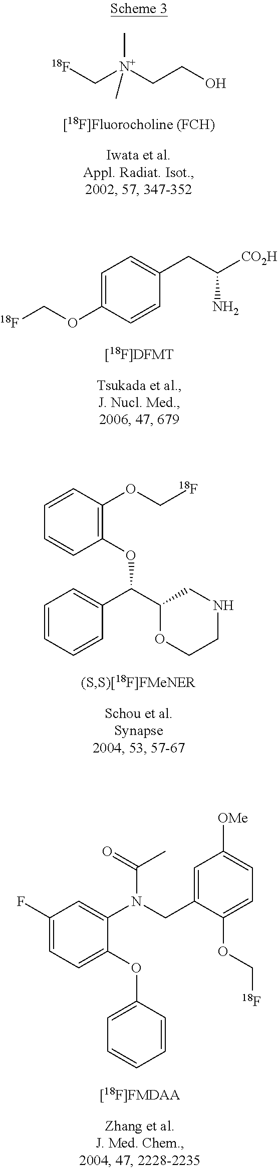

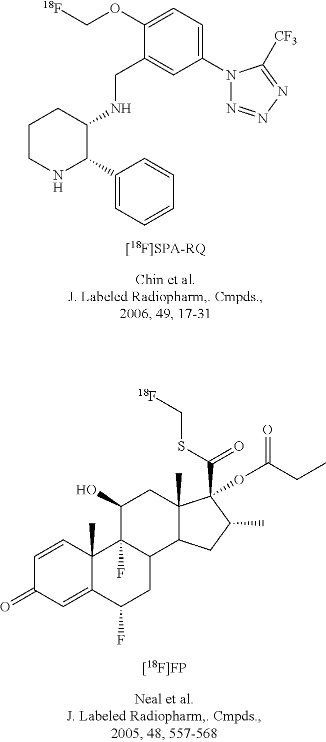

The radiosyntheses of [¹⁸F]fluoromethyl derivatives alkylated on a heteroatom are typically carried out via a two-step process as shown for a phenol derivative in Scheme 3. Briefly the process is: i) radiofluorination of a precursor (e.g. dibromomethane) to give a [¹⁸F]labeled alkylating agent (e.g. [¹⁸F]fluoromethyl bromide); ii) distillation of the [¹⁸F]labeled alkylating agent (e.g. [¹⁸F]fluoromethyl bromide) and iii) alkylation of heteroatom (e.g. phenol).

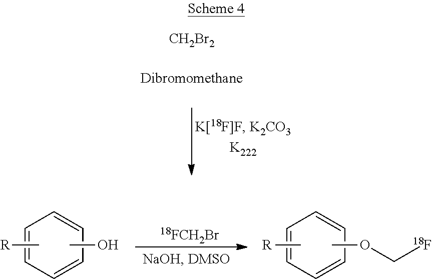

The synthesis of different [¹⁸F]fluoromethyl alkylating agents and their quick purification have been studied extensively by different research groups with the [¹⁸F]fluoromethyl bromide being the alkylating agent of choice. [¹⁸F]Fluoromethyl bromide was originally purified by gas chromatography (Bergman et al., Appl. Radiat. Isot., 2001, 54, 927-933), which involved dedicated equipment that can be expensive, cumbersome and require dedicated laboratory space. A simplified method using a distillation step through a series of four silica SPE cartridges has been described (Iwata et al., Appl. Radiat. Isot., 2002, 57, 347-352) and this method seems to be the method of choice used by numerous institutions and PET centres around the world.

Despite these improvements in the radiosynthesis of [¹⁸F]fluoromethyl bromide, there is a continued need for novel methods for improving and simplifying the radiosyntheses of F-18 radiolabeled compounds. This present application discloses improved methods for distilling radiolabeled fluoroalkylating agents, preferably [¹⁸F]fluoromethyl halides, more preferably [¹⁸F]fluoromethyl bromide.

### Problem to be Solved by the Invention and its Solution

Despite the aforementioned advances in simplified purification methods for different radiolabeled fluoroalkylating agents, there remains a need to improve and simplify the radiosynthesis of said [¹⁸F]fluoroalkylating agents. The current method widely-used to synthesize [¹⁸F]fluoromethyl bromide involves: 1) radiofluorination of the dibromomethane; 2) distillation of the [¹⁸F]fluoromethyl bromide through four silica SPE cartridges to remove the dibromomethane, which has a higher boiling point (100° C.) in comparison to fluoromethylbromide (8° C.). Four silica SPE cartridges are required to ensure that the level of dibromomethane co-distilled with the [¹⁸F]fluoromethyl bromide is kept to a minimum as the dibromomethane will compete with [¹⁸F]fluoromethyl bromide in the alkylation reaction affecting the yield and making the purification more complicated due to new impurities potentially being formed. One problem here is that the four silica SPE cartridges have to be connected to each other and they have to be completely sealed otherwise a loss of the desired [¹⁸F]fluoromethyl bromide product will be observed resulting in lower yields of the final [¹⁸F]fluoroalkylated product. We found that radiolabeled fluoroalkylating agents, preferably [¹⁸F]fluoromethyl halides, more preferably [¹⁸F]fluoromethyl bromide could be purified using a surprisingly simple solid phase C18 extraction (SPE) cartridges, optionally these SPE(s) can have Luer-locks to ensure a sealed system, whereby the amount of dibromomethane co-distilling with the [¹⁸F]fluoromethyl bromide was reduced considerably in comparison to analogous runs using the commonly used four silica SPE cartridges method of purification.

As a preferred solution the invention provides for a solid phase cartridge or column filled with modified silica or alumina gel/resin. Preferably said modified gel/resin is a reversed phase material. More preferably said modified gel/resin is a reversed phase material, wherein alkyl chains are covalently bond to the solid support. Even more preferably the alkyl chain is a C8 to C30 chain, more preferably a C8 to C20 chain, even more preferably a C15 to C20 chain, most preferred a C18 chain.

### SUMMARY

The invention relates to the methods referred to in the claims for the improved radiosynthesis of [¹⁸F]fluoromethyl bromide via a distillation step.

### DESCRIPTION

In a first aspect, the invention is directed to methods for the purification of compounds of formula (I)

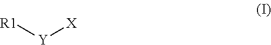

wherein
   R1 is Halogen or sulfonate,
   X is Fluorine atom (F),
   Y is CH₂, CHD, or CD₂, and
   D stands for Deuterium,
      comprising the step:
Purification of compound of formula (I) by distillation through at least one solid phase extraction (SPE) cartridge containing a stationary phase selected from the group comprising a C8 to C30 alkyl chain, more preferably a C8 to C20 alkyl chain, even more preferably a C15 to C20 alkyl chain, most preferred a C18 alkyl chain.

Preferably, in a first aspect, the invention is directed to methods for the purification of compounds of formula (I)

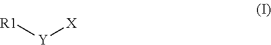

wherein
   R1 is Halogen or sulfonate,
   X is Fluorine atom (F),
   Y is CH₂, CHD, or CD₂, and
   D stands for Deuterium,
      comprising the step:
Purification of compound of formula (I) by distillation through at least one solid phase extraction (SPE) cartridge containing a stationary phase selected from the group comprising C30, C20, C18 and tC18, C15, and C8.

More preferably, in a first aspect, the invention is directed to methods for the purification of compounds of formula (I)

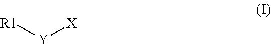

wherein
   R1 is Halogen or sulfonate,
   X is Fluorine atom (F),
   Y is CH₂, CHD, or CD₂, and
   D stands for Deuterium,
      comprising the step:
Purification of compound of formula (I) by distillation through at least one solid phase extraction (SPE) cartridge containing a stationary phase selected from the group comprising C30, C18 and tC18.

Preferably, Fluorine atom (F) is a ¹⁸F or ¹⁹F Fluorine isotope. More preferably, Fluorine atom (F) is a ¹⁸F Fluorine isotope.

Preferably, Y is CH₂ or CD₂. More preferably, Y is CH₂.

D stands for Deuterium.

Preferably, Halogen is chloro, bromo or iodo, and sulfonate is mesylate, toyslate, triflate or nosylate. More preferably, R1 is bromo or iodo.

Preferably, the solid phase extraction (SPE) cartridge containing a stationary phase is selected from the group comprising C18 and tC18.

Distillation is conducted by solid-phase-extraction using one (1) to five (5) SPE cartridge(s) containing a stationary phase selected from the group comprising a C8 to C30 alkyl chain, more preferably a C8 to C20 alkyl chain, even more preferably a C15 to C20 alkyl chain, most preferred a C18 alkyl chain.

Preferably, distillation is conducted by solid-phase-extraction using one (1) to five (5) SPE cartridge(s) containing a stationary phase selected from the group comprising C30, C20, C18 and tC18, C15 and C8.

More preferably, distillation is conducted by solid-phase-extraction using one (1) to five (5) SPE cartridge(s) containing a stationary phase selected from the group comprising C30, C18, and tC18. Preferably, distillation is conducted by solid-phase-extraction using one (1) to four (4) SPE cartridge(s), even more preferably one (1) to two (2), even more preferably one (1) SPE cartridge.

In a first embodiment, the invention is directed to a compound of formula (I) wherein the Fluorine atom (F) is a ¹⁸F fluorine isotope.

In a second embodiment, the invention is directed to a compound of formula (I) wherein the Fluorine atom (F) is a ¹⁹F fluorine isotope.

Preferably, compound of Formula (I) is selected from
bromofluoromethane (FCH₂Br), bromo[¹⁸F]fluoromethane ([¹⁸F]FCH₂Br), fluoroiodomethane (FCH₂I), [¹⁸F]fluoroiodomethane ([¹⁸F]FCH₂I) or their deuterated derivatives:
deuterated bromo[¹⁸F]fluoromethane ([¹⁸F]FCD₂Br), deuterated bromofluoromethane (FCD₂Br), monodeuterobromofluoromethane (FCHDBr), monodeutero-bromo[¹⁸F]fluoromethane ([¹⁸F]FCHDBr), deuterated fluoroiodomethane (FCD₂I), deuterated [¹⁸F]fluoroiodomethane ([¹⁸F]FCD₂I), monodeuterofluoroiodomethane (FCHDI), or monodeutero[¹⁸F]fluoroiodomethane ([¹⁸F]FCHDI).

Preferably, compound of Formula (I) is bromo[¹⁸F]fluoromethane ([¹⁸F]FCH₂Br) or bromofluoromethane (FCH₂Br).

Preferably, the invention is directed to methods for the purification of compounds of formula (I)

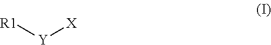

wherein
   R1 is bromo,
   X is ¹⁸F Fluorine isotope, and
   Y is CH₂,
      comprising the step:
Purification of compound of formula (I) by distillation through one (1) to four (4) solid phase extraction (SPE) cartridges containing a stationary phase selected from the group comprising C18 and tC18.

Embodiments and preferred features can be combined together and are within the scope of the invention.

In a second aspect, the invention is directed to methods for obtaining purified compounds of formula (I)

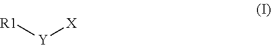

wherein
   R1 is Halogen or sulfonate,
   X is Fluorine atom (F),
   Y is CH₂, CHD, or CD₂, and
   D stands for Deuterium,
      comprising the steps:
Fluorination of compound of formula (II) with Fluorine atom (F) containing moiety for obtaining a compound of formula (I)
wherein compounds of formula (II) is

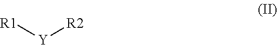

wherein
   R1 is a leaving group selected from the group of Halogen or sulfonate,
   R2 is a leaving group selected from the group of Halogen or sulfonate,
   Y is CH₂, CHD or CD₂ and
   D stands for Deuterium,
Purification of compound of formula (I) by distillation through at least one solid phase extraction (SPE) cartridges containing a stationary phase selected from the group comprising a C8 to C30 alkyl chain, more preferably a C8 to C20 alkyl chain, even more preferably a C15 to C20 alkyl chain, most preferred a C18 alkyl chain.

Preferably, in a second aspect, the invention is directed to methods for obtaining purified compounds of formula (I)

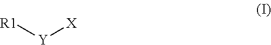

wherein
   R1 is Halogen or sulfonate,
   X is Fluorine atom (F),
   Y is CH₂, CHD, or CD₂, and
   D stands for Deuterium,
      comprising the steps:
Fluorination of compound of formula (II) with Fluorine atom (F) containing moiety for obtaining a compound of formula (I)
wherein compounds of formula (II) is

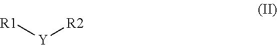

wherein
   R1 is a leaving group selected from the group of Halogen or sulfonate,
   R2 is a leaving group selected from the group of Halogen or sulfonate,
   Y is CH₂, CHD or CD₂ and
   D stands for Deuterium,
Purification of compound of formula (I) by distillation through at least one solid phase extraction (SPE) cartridge containing a stationary phase selected from the group comprising C30, C20, C18 and tC18, C15 and C8.

More preferably, in a second aspect, the invention is directed to methods for obtaining purified compounds of formula (I)

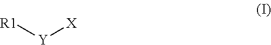

wherein
   R1 is Halogen or sulfonate,
   X is Fluorine atom (F),
   Y is CH₂, CHD, or CD₂, and
   D stands for Deuterium,
      comprising the steps:
Fluorination of compound of formula (II) with Fluorine atom (F) containing moiety for obtaining a compound of formula (I)
wherein compounds of formula (II) is

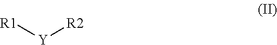

wherein
   R1 is a leaving group selected from the group of Halogen or sulfonate,
   R2 is a leaving group selected from the group of Halogen or sulfonate,
   Y is CH₂, CHD or CD₂ and
   D stands for Deuterium,
Purification of compound of formula (I) by distillation through at least one solid phase extraction (SPE) cartridge containing a stationary phase selected from the group comprising C30, C18, and tC18.

Preferably, Fluorine atom (F) is a ¹⁸F or ¹⁹F Fluorine isotope. More preferably, Fluorine atom (F) is a ¹⁸F Fluorine isotope.

Preferably, Y is CH₂ or CD₂. More preferably, Y is CH₂.

D stands for Deuterium.

Preferably, Halogen is chloro, bromo or iodo, and sulfonate is mesylate, toyslate, triflate or nosylate. More preferably, R1 is bromo or iodo.

Preferably, the solid phase extraction (SPE) cartridge(s) contain(s) a stationary phase, which is selected from the group comprising C18, and tC18.

Preferably, compound of Formula (II) is selected from deuterated dibromomethane (CD₂Br₂), monodeuterodibromomethane (CHDBr₂), dibromomethane (CH₂Br₂), deuterated diiodomethane (CD₂I₂), monodeuterodiiodomethane (CHDI₂), and diiodomethane (CH₂I₂). More preferably, compound of Formula (II) is deuterated dibromomethane (CD₂Br₂) or dibromomethane (CH₂Br₂).

The reagents, solvents and conditions which can be used for this fluorination are common and well-known to the skilled person in the field. See, e.g., J. Fluorine Chem., 27 (1985):177-191.

Preferably, the solvent used in the present method is DMF, DMSO, acetronitrile, DMA, or mixture thereof, preferably the solvent is acetonitrile.

Preferably, the Fluorine atom (F) containing moiety comprising ¹⁸F can be chelated complexes known to those skilled in the art, e.g. 4,7,13,16,21,24-Hexaoxa-1,10-diazabicyclo[8.8.8]-hexacosane K¹⁸F (crown ether salt Kryptofix K¹⁸F), 18-crown-6 ether salt K¹⁸F, K¹⁸F, H¹⁸F, KH¹⁸F₂, Rb¹⁸F, Cs¹⁸F, Na¹⁸F, or tetraalkylammonium salts of ¹⁸F known to those skilled in the art, e.g. [¹⁸F]tetrabutylammonium fluoride, or tetraalkylphosphonium salts of ¹⁸F known to those skilled in the art, e.g. [¹⁸F]tetrabutylphosphonium fluoride. Most preferably, the Fluorine atom (F) containing moiety is Cs¹⁸F, K¹⁸F, H¹⁸F, or KH¹⁸F₂.

More preferably, Fluorine atom (F) containing moiety comprises ¹⁹F. Even more preferably, the Fluorine atom (F) containing moiety is 4,7,13,16,21,24-Hexaoxa-1,10-diazabicyclo[8.8.8]-hexacosane KF (crownether salt Kryptofix KF), 1,4,7,10,13,16-hexaoxacyclooctadecane KF, KF, tetrabutylammonium fluoride, tetrabutylammonium dihydrogen trifluoride.

Preferably, the invention is directed to methods for obtaining purified compounds of formula (I)

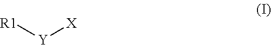

wherein
   R1 is bromo,
   X is ¹⁸F Fluorine isotope, and
   Y is CH₂,
      comprising the steps:
Fluorination of compound of formula (II) with Fluorine atom (F) containing moiety comprising ¹⁸F for obtaining a compound of formula (I)
wherein compounds of formula (II) is

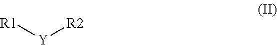

wherein
   R1 is bromo,
   R2 is bromo, and
   Y is CH₂,
Purification of compound of formula (I) by distillation through one (1) to four (4) solid phase extraction (SPE) cartridge(s) containing a stationary phase selected from the group comprising C18, and tC18.

In a third aspect, the invention is directed to a composition comprising compounds of the formula (I) obtained by the methods of the first aspect or the second aspect and pharmaceutically acceptable carrier or diluent.

The person skilled in the art is familiar with auxiliaries, vehicles, excipients, diluents, carriers or adjuvants which are suitable for the desired pharmaceutical formulations, preparations or compositions on account of his/her expert knowledge.

In a fourth aspect, the present invention provides a kit comprising a sealed vial containing a predetermined quantity of
the compounds of Formula (II) and;
solid phase extraction (SPE) cartridge(s) containing a stationary phase selected from the group comprising a C8 to C30 alkyl chain, more preferably a C8 to C20 alkyl chain, even more preferably a C15 to C20 alkyl chain, most preferred a C18 alkyl chain.

Preferably, in a fourth aspect, the present invention provides a kit comprising a sealed vial containing a predetermined quantity of
the compounds of Formula (II) and;
solid phase extraction (SPE) cartridge(s) containing a stationary phase selected from the group comprising C30, C20, C18 and tC18, C15, and C8.

More preferably, in a fourth aspect, the present invention provides a kit comprising a sealed vial containing a predetermined quantity of
the compounds of Formula (II) and;
solid phase extraction (SPE) cartridge(s) containing a stationary phase selected from the group comprising C30, C18 and tC18.

Preferably, the kit comprises one (1) to five (5) SPE cartridge(s) containing a stationary phase selected from the group comprising C30, C18, and tC18. More preferably, the kit comprises one (1) to four (4) SPE cartridge(s), even more preferably one (1) to two (2), even more preferably one (1) SPE cartridge. Preferably, the solid phase extraction (SPE) cartridge containing a stationary phase is selected from the group comprising C18 and tC18.

Optionally the kit comprises a pharmaceutically acceptable carrier, diluent, excipient or adjuvant.

### DEFINITIONS

The terms used in the present invention are defined below but are not limiting the invention's scope.

Solid-phase extraction (SPE) is an extraction method that uses a solid phase and a liquid phase to isolate analytes or products of a pre-defined type, e.g. lipophilic, hydrophilic, basic, or acidic ones, from a solution containing different species. The general method is to load a solution onto the SPE phase and trap the desired analyte or product, wash away undesired components. Then the desired analyte or product is eluted with a different solvent or solution and collected. Solid-phase extractions use the similar types of stationary phases that are used in liquid chromatography columns. The stationary phase is usually contained in a glass or plastic column above a frit or glass wool. Commercial SPE cartridges have 1-10 mL capacities and are discarded after use. Non-limiting examples of the stationary solid phases are: silica gel, modified silica gel, alumina, resins, polymers, copolymers or mixtures or layers thereof. In a more preferred embodiment, the stationary phase is selected from the group comprising silica, alumina A, alumina B, alumina N, magnesium silicate, magnesium oxide, zirconium oxide, C30, C18, tC18, C8, C4, C2, tC2, amino propyl (NH2), cyano propyl (CN), diol, hydroxyapatite, cellulose, graphitized carbon, weak cation exchange, medium cation exchange, strong cation exchange, weak anion exchange, medium anion exchange, strong anion exchange and polystyrene/divinylbenzene polymers or copolymers thereof.

Preferably the “solid-phase extraction (SPE) cartridge(s)” pursuant to the invention is/are filled with modified silica or alumina gel/resin. Preferably said modified gel/resin is a reversed phase material. Preferably said modified gel/resin is a reversed phase material, wherein alkyl chains are covalently bond to the solid support. Preferably the alkyl chain is a C8 to C30 chain, more preferably a C8 to C20 chain, even more preferably a C15 to C20 chain, most preferred a C18 chain.

More preferably, the “solid-phase extraction (SPE) cartridge(s)” pursuant to the invention is/are filled with modified silica or alumina gel/resin. Preferably said modified gel/resin is a reversed phase material. Preferably said modified gel/resin is a reversed phase material, wherein alkyl chains are covalently bond to the solid support. Preferably the alkyl chain is C30, C20, C18 and tC18, C15 and C8.

The entire disclosure(s) of all applications, patents and publications, cited herein are incorporated by reference herein.

The following examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

### General Synthesis of F-18 Compounds

The radiofluorination reaction can be carried out, for example in a typical reaction vessel (e.g. Wheaton vial) which is known to someone skilled in the art or in a microreactor. The reaction can be heated by typical methods, e.g. oil bath, heating block or microwave. The radiofluorination reactions are carried out in dimethylformamide with potassium carbonate as base and “Kryptofix” as crownether. But also other solvents can be used which are well known to experts. These possible conditions include, but are not limited to: dimethylsulfoxide and acetonitrile as solvent and tetraalkyl ammonium and tetraalkyl phosphonium carbonate as base. Water and/or alcohol can be involved in such a reaction as co-solvent. The radiofluorination reactions are conducted for one to 60 minutes. Preferred reaction times are five to 50 minutes. Further preferred reaction times are 10 to 40 min. This and other conditions for such radiofluorination are known to experts (Coenen, Fluorine-18 Labeling Methods: Features and Possibilities of Basic Reactions, (2006), in: Schubiger P. A., Friebe M., Lehmann L., (eds), PET-Chemistry—The Driving Force in Molecular Imaging. Springer, Berlin Heidelberg, pp. 15-50). The radiofluorination can be carried out in a “hot-cell” and/or by use of a module (review: Krasikowa, Synthesis Modules and Automation in F-18 labeling (2006), in: Schubiger P. A., Friebe M., Lehmann L., (eds), PET-Chemistry—The Driving Force in Molecular Imaging. Springer, Berlin Heidelberg, pp. 289-316), which allows an automated or semi-automated synthesis.

The radiofluorination reaction can be carried out, for example in a typical reaction vessel (e.g. Wheaton vial) which is known to someone skilled in the art or in a microreactor. The reaction can be heated by typical methods, e.g. oil bath, heating block or microwave. The radiofluorination reactions are carried out in dimethylformamide with potassium carbonate as base and “kryptofix” as crownether. But also other solvents can be used which are well known to experts. These possible conditions include, but are not limited to: acetonitrile, dimethylsulfoxide, sulfolane, dichloromethane, tetrahydrofuran, tertiary alcohols and o-dichlorobenzene as solvent and alkali metal with and without a suitable alkali metal chelating crown ether, tetraalkyl ammonium and tetraalkyl phosphonium carbonate as base. Water and/or alcohol can be involved in such a reaction as co-solvent. The radiofluorination reactions are conducted for one to 60 minutes. Preferred reaction times are five to 50 minutes. Further preferred reaction times are 10 to 40 min. This and other conditions for such radiofluorination are known to experts (Coenen, Fluorine-18 Labeling Methods: Features and Possibilities of Basic Reactions, (2006), in: Schubiger P. A., Friebe M., Lehmann L., (eds), PET-Chemistry—The Driving Force in Molecular Imaging. Springer, Berlin Heidelberg, pp. 15-50). The radiofluorination can be carried out in a “hot-cell” and/or by use of a module (eview: Krasikowa, Synthesis Modules and Automation in F-18 labeling (2006), in: Schubiger P. A., Friebe M., Lehmann L., (eds), PET-Chemistry—The Driving Force in Molecular Imaging. Springer, Berlin Heidelberg, pp. 289-316) which allows an automated or semi-automated synthesis.

### General Synthesis of F-18 Compounds

The ¹⁸F-compounds were synthesized by reaction of precursors of Formula II with [¹⁸F]fluoride to give ¹⁸F labeled intermediates of Formula I, which were then reacted with precursors of Formula III to give the desired product of Formula IV as shown in Scheme 3.

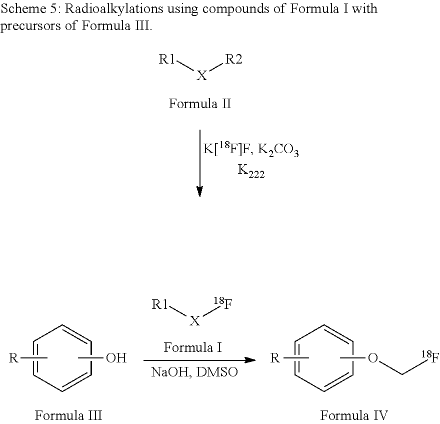

One example of this alkylation is illustrated in Scheme 3 with [¹⁸F]fluoromethyl bromide (Formula I) reacting with Tyrosine (Formula II) to give the desired product of O-fluoromethyltyrosine (Formula IV).

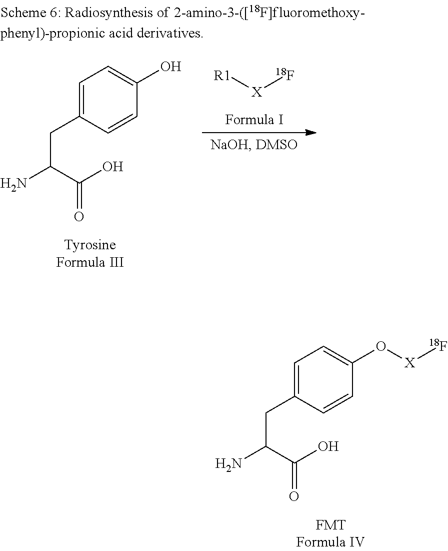

### EXPERIMENTAL SECTION

### Abbreviations

| | |
|---|---|
| CH₂Br₂ | Dibromomethane |
| DMSO | Dimethylsulfoxide |
| GC | Gas Chromatography |
| K₂₂₂ | Kryptofix 2.2.2 |
| K₂CO₃ | Potassium carbonate |
| min | minute |
| ml | millilitre |
| QMA | Quaternary Methyl Ammonium |
| SPE | Solid Phase Extraction |
| TBAOH | Tetrabutylammonium hydroxide |
| U-HPLC | Ultra High Pressure Liquid |
| | Chromatography |

### General:

All solvents and chemicals were obtained from commercial sources and used without further purification. Anhydrous solvents and inert atmosphere (nitrogen or argon) were used if not stated otherwise. The preceding table lists the abbreviations used in this paragraph and in the Examples sections as far as they are not explained within the text body.

### Reaction Conditions

All radiosyntheses were carried out using the same GE MX automated synthesizer fitted with silicon tubings (1.5×3 mm) and using the manifolds used in the 2-[¹⁸F]fluorodeoxyglucose radiosynthesis (known to those skilled in the art). The reactors were 6 ml reactor vials having a 20 mm crimp top. These GE MX automated synthesizer has a low and high flow; the low flow was measured to be 39-49 ml/min (exact flow is shown for each experiments in Table 1).

### Fluorination Conditions:

[¹⁸F]Fluoride was immobilized on a preconditioned QMA (Waters) cartridge. The [¹⁸F]fluoride was eluted using either a solution of:
i) K₂CO₃ (2.7 mg) in 50 μl water and K₂₂₂ (15 mg) in 950 μl acetonitrile or
ii) 75 mM tetrabutylammonium hydroxide (TBAOH) solution in water/ethanol (9:1) (750 μl)

This eluted solution was dried under vacuum and additional acetonitrile was added and the drying step was repeated. A solution of dibromomethane (CH₂Br₂; 300 μl) in acetonitrile (2700 μl) was added and heated at 120° C. for 5 min. The bromo[¹⁸F]fluoromethane was distilled at 120° C. into vials of DMSO (2 ml) connected in series (max. 4) at ambient temperature; this distillation of [¹⁸F]fluoromethyl bromide was carried out with slightly different nitrogen flows (see Table 1) through the following different SPEs:
i) 3 or 4× silica SPE cartridges (standard known method Iwata et al., Appl. Radiat. Isot., 2002, 57, 347-352)
ii) 4× Alltech Maxi Clean Silica (900 mg)
iii) 1× Luknova Silica Flash Cartridge 4 g
iv) 4× C18 environmental SPE (820 mg)
v) 1 or 2× C18 Flash cartridge 6 ml (1 g)
vi) 4× tC18 Plus Environmental SPE (400 mg)
vii) 1× C18 Flash cartridge 6 ml (0.5 g)
viii) 4× C8 Plus SPE (0.4 g)

The DMSO solution used for trapping the radioactive product was analyzed for the:
i) Yield of the [¹⁸F]fluoromethyl bromide product
ii) Purity of the [¹⁸F]fluoromethyl bromide product (U-HPLC Dionex Ultimate 3000; column ACE 3 C18 50 mm×4.6 mm 3 μm, Solvent A=Water+0.027% H₂SO₄, Solvent B=Acetonitrile+0.027% H₂SO₄; Gradient: 0-3 min 100% A, 3-7 min 100% A to 82.9% A, 7-7.1 min from 82.9% A to 10% A; Flow: 2 ml/min
iii) Amount of the dibromomethane precursor breaking through (GC Headspace: Agilent G1888, Agilent Technologies 6890N ; Column: J&W123-1334 DB 624 Agilent Technologies; 50 μl of the DMSO trapping solution was injected into a 20 ml Headspace-Vial, initial injector temperature 130° C., column temperature 40° C. for 8 mins, then 10° C./min to 150° C., 150° C. for 4 min, split ratio 1:1, total flow 6.4 ml/min of nitrogen; FID Detector: temperature 250° C.).

TABLE 1
Summary of the radiosyntheses of [¹⁸F]fluoromethyl bromide

| | | | | | Amount |
|---|---|---|---|---|---|
| | | No. of | Flow | Yield | CH₂Br₂ |
| SPE | Elution | SPEs | (ml/min) | [¹⁸F]FCH₂Br | (μg/ml) |
| Silica | K₂CO₃/K₂₂₂ | 3 | 40.5 | 28.5% | 72.40 |
| (690 mg) | | | | | |
| Silica | K₂CO₃/K₂₂₂ | 4 | 42.3 | 29.1% | 4.70 |
| (690 mg) | | | | | |
| Silica | K₂CO₃/K₂₂₂ | 4 | 45.0 | 22.6% | 12.59 |
| (900 mg) | | | | | |
| Silica (4 g) | K₂CO₃/K₂₂₂ | 1 | 45.0 | 34.2% | 52.50 |
| C18 | K₂CO₃/K₂₂₂ | 4 | 45.0 | 33.0% | 0.00 |
| (820 mg) | | | | | |
| C18 | K₂CO₃/K₂₂₂ | 4 | 42.5 | 31.6% | 0.00 |
| (820 mg) | | | | | |
| C18 | K₂CO₃/K₂₂₂ | 4 | 39.5 | 30.3% | 0.00 |
| (820 mg) | | | | | |
| C18 Flash | K₂CO₃/K₂₂₂ | 2 | 40.2 | 21.6% | 0.00 |
| (1 g) | | | | | |
| C18 Flash | K₂CO₃/K₂₂₂ | 1 | 39.9 | 18.7% | 0.09 |
| (1 g) | | | | | |
| tC18 | K₂CO₃/K₂₂₂ | 4 | 42 | 32.3% | 0.00 |
| tC18 | K₂CO₃/K₂₂₂ | 4 | 49 | 23.4% | 0.31 |
| tC18 | K₂CO₃/K₂₂₂ | 4 | 39.6 | 23.5% | 0.00 |
| C18 Flash | TBAOH | 1 | 39.6 | 19.0% | 0.00 |
| (0.5 g) | | | | | |
| C18 Flash | TBAOH | 1 | — | 24.7% | 0.00 |
| (0.5 g) | | | | | |
| C8 (0.4 g) | K₂CO₃/K₂₂₂ | 4 | 39.4 | 25.2% | 181.46 |

Silica (690 mg) = Silica SPE (Waters WAT020520)
Silica (900 mg) = Alltech Maxi Clean Silica (SI) 900 mg (Part. No. 20988)
Silica (4 g) = Luknova Silica Flash Cartridge 4 g (Part No. FC003004)
C18 (820 mg) = C18 environmental SPE (Waters WAT023635)
tC18 = tC18 Plus Environmental SPE (Waters WAT036810)
C18 Flash (1 g) = C18 Flash cartridge 6 ml 1 g (Macherey-Nagel 730005)
C18 Flash (0.5 g) = C18 Flash cartridge 6 ml 0.5 g (Macherey-Nagel 732999)
C8 (0.4 g) = C8 Plus SPE (Waters WAT036775)

The surprising result of these distillations is that the use of C18 SPEs significantly reduce the amount of dibromomethane breaking through into the DMSO solution—any breakthrough of dibromomethane will result in additional side reactions as dibromomethane is also an alkylating reagents, and thus, the need for better purification methods may be required.

1. A method for the purification of compounds of formula (I) 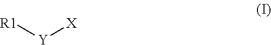
wherein
R1 is Halogen or sulfonate,
X is Fluorine atom (F),
Y is CH₂, CHD, or CD₂, and
D stands for Deuterium,
comprising the step:
Purification of compound of formula (I) by distillation through at least one solid phase extraction (SPE) cartridge containing a stationary phase selected from the group comprising a C8 to C30 alkyl chain, more preferably a C8 to C20 alkyl chain, even more preferably a C15 to C20 alkyl chain, most preferred a C18 alkyl chain.

2. The method according to claim 1 for the purification of compounds of formula (I) 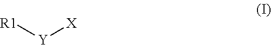
wherein
R1 is Halogen or sulfonate,
X is Fluorine atom (F),
Y is CH₂, CHD, or CD₂, and
D stands for Deuterium,
comprising the step:
Purification of compound of formula (I) by distillation through at least one solid phase extraction (SPE) cartridge containing a stationary phase selected from the group comprising C30, C20, C18 and tC18, C15 and C8.

3. The method according to claim 1 for the purification of compounds of formula (I) 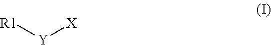
wherein
R1 is Halogen or sulfonate,
X is Fluorine atom (F),
Y is CH₂, CHD, or CD₂, and
D stands for Deuterium,
comprising the step:
Purification of compound of formula (I) by distillation through at least one solid phase extraction (SPE) cartridge containing a stationary phase selected from the group comprising C30, C18 and tC18.

4. The method according to claim 1, wherein the Fluorine atom (F) is a ¹⁸F Fluorine isotope and Y is CH₂ or CD₂.

5. The method according to claim 1, wherein the solid phase extraction (SPE) cartridge(s) containing a stationary phase is selected from the group comprising C18 and tC18.

6. The method according to claim 1, wherein one (1) to five (5) solid phase extraction (SPE) cartridge(s) are used.

7. The method according to the claim 1, wherein compound of Formula (I) is selected from
bromofluoromethane (FCH₂Br), bromo[¹⁸F]fluoromethane ([¹⁸F]FCH₂Br), fluoroiodomethane (FCH₂I), [¹⁸F]fluoroiodomethane ([¹⁸F]FCH₂I) or their deuterated derivatives:
deuterated bromo[¹⁸F]fluoromethane ([¹⁸F]FCD₂Br), deuterated bromofluoromethane (FCD₂Br), monodeuterobromofluoromethane (FCHDBr), monodeutero-bromo[¹⁸F]fluoromethane ([¹⁸F]FCHDBr), deuterated fluoroiodomethane (FCD₂I), deuterated [¹⁸F]fluoroiodomethane ([¹⁸F]FCD₂I), monodeuterofluoroiodomethane (FCHDI), or monodeutero[¹⁸F]fluoroiodomethane ([¹⁸F]FCHDI).

8. A method for obtaining purified compounds of formula (I) 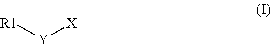
wherein
R1 is Halogen or sulfonate,
X is Fluorine atom (F),
Y is CH₂, CHD, or CD₂, and
D stands for Deuterium,
comprising the steps:
Fluorination of compound of formula (II) with Fluorine atom (F) containing moiety for obtaining a compound of formula (I)
wherein compound of formula (II) is 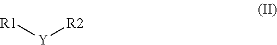
wherein
R1 is a leaving group selected from the group of Halogen or sulfonate,
R2 is a leaving group selected from the group of Halogen or sulfonate,
Y is CH₂, CHD or CD₂ and
D stands for Deuterium,
Purification of compound of formula (I) by distillation through at least one solid phase extraction (SPE) cartridge containing a stationary phase selected from the group comprising a C8 to C30 alkyl chain, more preferably a C8 to C20 alkyl chain, even more preferably a C15 to C20 alkyl chain, most preferred a C18 alkyl chain.

9. The method according to claim 8 for obtaining purified compounds of formula (I) 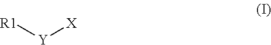
wherein
R1 is Halogen or sulfonate,
X is Fluorine atom (F),
Y is CH₂, CHD, or CD₂, and
D stands for Deuterium,
comprising the steps:
Fluorination of compound of formula (II) with Fluorine atom (F) containing moiety for obtaining a compound of formula (I)
wherein compound of formula (II) is 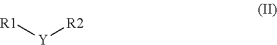
wherein
R1 is a leaving group selected from the group of Halogen or sulfonate,
R2 is a leaving group selected from the group of Halogen or sulfonate,
Y is CH₂, CHD or CD₂ and
D stands for Deuterium,
Purification of compound of formula (I) by distillation through at least one solid phase extraction (SPE) cartridge containing a stationary phase selected from the group comprising C30, C20, C18 and tC18, C15 and C8.

10. The method according to claim 8 for obtaining purified compounds of formula (I) 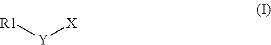
wherein
R1 is Halogen or sulfonate,
X is Fluorine atom (F),
Y is CH₂, CHD, or CD₂, and
D stands for Deuterium,
comprising the steps:
Fluorination of compound of formula (II) with Fluorine atom (F) containing moiety for obtaining a compound of formula (I)
wherein compound of formula (II) is 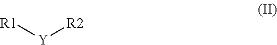
wherein
R1 is a leaving group selected from the group of Halogen or sulfonate,
R2 is a leaving group selected from the group of Halogen or sulfonate,
Y is CH₂, CHD or CD₂ and
D stands for Deuterium,
Purification of compound of formula (I) by distillation through at least one solid phase extraction (SPE) cartridge containing a stationary phase selected from the group comprising C30, C18, and tC18.

11. The method according to claim 8, wherein
compound of Formula (II) is selected from deuterated dibromomethane (CD₂Br₂), monodeuterodibromomethane (CHDBr₂), dibromomethane (CH₂Br₂), deuterated diiodomethane (CD₂I₂), monodeuterodiiodomethane (CHDI₂), and diiodomethane (CH₂I₂) and
compound of Formula (I) is selected from bromofluoromethane (FCH₂Br), bromo[¹⁸F]fluoromethane ([¹⁸F]FCH₂Br), fluoroiodomethane (FCH₂I), [¹⁸F]fluoroiodomethane ([¹⁸F]FCH₂I) or their deuterated derivatives:
deuterated bromo[¹⁸F]fluoromethane ([¹⁸F]FCD₂Br), deuterated bromofluoromethane (FCD₂Br), monodeuterobromofluoromethane (FCHDBr), monodeutero-bromo[¹⁸F]fluoromethane ([¹⁸F]FCHDBr), deuterated fluoroiodomethane (FCD₂I), deuterated [¹⁸F]fluoroiodomethane ([¹⁸F]FCD₂I), monodeuterofluoroiodomethane (FCHDI), or monodeutero[¹⁸F]fluoroiodomethane ([¹⁸F]FCHDI).

12. A kit comprising a sealed vial containing a predetermined quantity of
the compounds of Formula (II) as defined in claim 8 and;
solid phase extraction (SPE) cartridge(s) containing a stationary phase selected from the group comprising a C8 to C30 alkyl chain, more preferably a C8 to C20 alkyl chain, even more preferably a C15 to C20 alkyl chain, most preferred a C18 alkyl chain.

13. The kit according to claim 12 comprising a sealed vial containing a predetermined quantity of
the compounds of Formula (II) and;
solid phase extraction (SPE) cartridge(s) containing a stationary phase selected from the group comprising C30, C20, C18 and tC18, C15 and C8.

14. The kit according to claim 12 comprising a sealed vial containing a predetermined quantity of
the compounds of Formula (II) and;
solid phase extraction (SPE) cartridge(s) containing a stationary phase selected from the group comprising C30, C18 and tC18.
